(19) United States
(12) Patent Application Publication
(10) Pub. No.: US 2006/0042940 A1
(43) Pub. Date: Mar. 2, 2006

(54) Alcohol concentration detector, method of detecting alcohol concentration therewith and process for producing alcohol concentration detection sensor

(76) Inventors: Toshiaki Kawanishi, Saitama (JP); Kiyoshi Yamagishi, Saitama (JP); Takayuki Takahata, Saitama (JP)

Correspondence Address: Kent E Baldauf, 700 Koppers Building, 436 Seventh Avenue, Pittsburgh, PA, 15219-1818, US

(21) Appl. No.: 10/529,709

(22) PCT Filed: Sep. 30, 2003
(86) PCT No.: PCT/JP03/12504

§ 371 Date: Mar. 29, 2005

(30) Foreign Application Priority Data
Sep. 30, 2002 (JP) 2002-286668

(51) Int. Cl. G01N 33/487

(52) U.S. Cl. 204/403.01

(57) ABSTRACT

It is an object to provide an alcohol concentration detecting apparatus which is small-sized and compact, can be installed everywhere and can have the degree of freedom of a design, has an excellent insulation between electrodes and is not influenced by a moisture, can carry out shielding in order not to be influenced by an electromagnetic wave generated from a body of a car or the like, and furthermore, can execute an accurate measurement for an alcohol concentration. Furthermore, it is an object to provide an alcohol concentration detecting method using the same apparatus, and an alcohol concentration detecting sensor manufacturing method. In an alcohol concentration detecting apparatus in which an alcohol concentration in the liquid to be inspected is detected by introducing a liquid to be inspected between electrodes of an alcohol concentration detecting sensor and by measuring a change in a specific inductive capacity of the liquid to be inspected between the electrodes with an oscillation frequency, the alcohol concentration detecting sensor comprises an alcohol concentration detecting sensor body including a base material resin film, an electrode wiring pattern formed on the base material resin film, and an insulating resin covering a surface of the electrode wiring pattern.

### TECHNICAL FIELD

The present invention relates to an alcohol concentration detecting apparatus for detecting an alcohol concentration in a liquid to be inspected, for example, a gasoline and an alcohol concentration detecting method using the same, and an alcohol concentration detecting sensor manufacturing method.

### BACKGROUND ART

Conventionally, for example, a so-called high-octane gasoline having an octane value increased such as lead or a benzene based compound, or a gasoline into which an antiknocking agent such as methyl tertiary butyl ether or methyl-t-butyl ether (MTBE) is mixed has been used for a car or the like in order to prevent knocking.

However, there is a possibility that the lead, the benzene based compound or the like might influence an environment. Moreover, it is said that the methyl tertiary butyl ether or the methyl-t-butyl ether (MTBE) is cancer-causing. For this reason, it has been desired to develop the high-octane gasoline and an antiknocking agent in place of the antiknocking agent constituted by the methyl tertiary butyl ether or the methyl-t-butyl ether (MTBE).

Therefore, it has been proposed that alcohol, for example, ethanol is added, as the antiknocking agent, in an amount of approximately 10 to 15% to a gasoline.

However, such ethanol is added so that a torque is reduced. By excessively adding a gasoline corresponding to the amount of addition of the ethanol, therefore, it is necessary to cause the torque to be constant.

For this reason, it has been desired to detect the concentration of alcohol contained in a gasoline.

As a method of detecting the concentration of alcohol, conventionally, an optical alcohol concentration measuring apparatus for detecting the concentration of alcohol by utilizing the refractive index of a light has been disclosed as in Japanese Laid-Open Patent Publication No. Hei 5(1993)-223733 (see paragraphs (0017) to (0030) and FIG. 1) (which will be hereinafter referred to as “Patent Document 1”).

More specifically, in an optical alcohol concentration measuring apparatus 100 in the Patent Document 1, a light which is transmitted from a light emitting portion 102 through a liquid and has a wavelength with such a property that an absorption into alcohol such as ethanol is hard to perform is received by a first light receiving portion 104. Then, a detection signal corresponding to an alcohol concentration in the liquid is output as shown in FIG. 19.

Moreover, a light, which is transmitted from a second light emitting portion 106 through the liquid and has another wavelength with such a property that the absorption into the alcohol is easy, is received by a second light receiving portion 108. Then, a detection signal corresponding to the alcohol concentration in the liquid is output.

Consequently, the detection signal sent from the first light receiving portion 104 is compared with the detection signal sent from the second light receiving portion 108 and the alcohol concentration in the liquid is measured in a measuring portion 110.

As described in “Electrostatic Capacitance Type Alcohol Concentration Sensor” (see Norio Mima, Ikuo Hayashi, Ichiro Hosoya, The Society of Automotive Engineers of Japan, Annual Congress Preliminary Printing Collection 936, 1993-10, pages 257 to 260) (which will be hereinafter referred to as “Non-Patent Document 1”), conventionally, an electrostatic capacitance type alcohol concentration sensor has been proposed.

In the Non-Patent Document 1, there has been proposed a method of measuring the concentration of methanol mixed into a gasoline from an electrostatic capacitance between electrodes at an oscillation frequency by utilizing a difference in a specific inductive capacity between the gasoline and the methanol (the gasoline has a specific inductive capacity of 2 and the methanol has a specific inductive capacity of 33.6), thereby detecting the concentration of the methanol.

An electrostatic capacitance type alcohol concentration sensor 200 according to the Non-Patent Document 1 has such a structure that an outer electrode 204 and a center electrode 206 are attached through an insulating resin 208 into a housing 202 as shown in FIG. 20.

Since the optical alcohol concentration measuring apparatus according to the Patent Document 1 utilizes a transmitted light, however, it is easily influenced by the composition of a gasoline. For example, in the case in which a liquid to be inspected is not transparent due to an impurity, moreover, a measurement cannot be carried out or an accurate measurement cannot be performed.

In the electrostatic capacitance type alcohol concentration sensor utilizing an electrostatic capacitance according to the Non-Patent Document 1, furthermore, a moisture is apt to enter alcohol and a short circuit is generated between electrodes if the moisture, an electrolyte or the like is present between the electrodes. Accordingly, an insulating treatment for the surface of the electrode is required and a structure thereof is complicated.

In this case, an electrostatic capacitance Cₛ is expressed in the following equation.
Cₛ=ε₀(S/D)(εra(α/100εrg(1−α/100)) Equation 1

Herein, S represents an opposed area of an electrode, D represents a distance between the electrodes, ε₀ represents a specific inductive capacity of a vacuum (8.854E-12F/m), εra represents a specific inductive capacity of alcohol, εrg represents a specific inductive capacity of a gasoline, and α represents an alcohol concentration (%).

As is apparent from the Equation, accordingly, it is preferable to increase the opposed area of the electrode to increase the electrostatic capacitance Cₛ in order to obtain the excellent results of the measurement. When the opposed area of the electrode is thus increased, however, the size of the electrostatic capacitance type alcohol concentration sensor itself is increased as in the Non-Patent Document 1. For this reason, handling, an application to a car and the like are also restricted in respect of a design.

In the electrostatic capacitance type alcohol concentration sensor according to the Non-Patent Document 1, furthermore, the sensor is to be connected to a body of a gasoline piping in a car or the like, for example. However, a noise such as an electromagnetic wave or the like which is generated from the body influences an alcohol concentration detecting circuit so that an accurate measurement cannot be carried out.

For this reason, an insulating structure is added to the connecting portion of the sensor and the piping or the whole large-sized sensor is to be put in an insulating shield container. Consequently, the apparatus becomes complicated and large-sized.

In consideration of such existing circumstances, it is an object of the present invention to provide an alcohol concentration detecting apparatus for detecting an alcohol concentration in a liquid to be inspected such as a gasoline which is small-sized and compact, can be installed everywhere and can have the degree of freedom of a design, has an excellent insulation between electrodes and is not influenced by a moisture, can carry out shielding in order not to be influenced by an electromagnetic wave generated from a body of a car or the like, and furthermore, can execute an accurate measurement for the alcohol concentration. Furthermore, it is an object to provide an alcohol concentration detecting method using the same apparatus, and an alcohol concentration detecting sensor manufacturing method.

### DISCLOSURE OF THE INVENTION

In order to solve the problems and to attain the object in the conventional art described above, the present invention has been made and provides an alcohol concentration detecting apparatus in which an alcohol concentration in the liquid to be inspected is detected by introducing a liquid to be inspected between electrodes of an alcohol concentration detecting sensor and by measuring a change in a specific inductive capacity of the liquid to be inspected between the electrodes with an oscillation frequency,
wherein the alcohol concentration detecting sensor comprises an alcohol concentration detecting sensor body including a base material resin film, an electrode wiring pattern formed on the base material resin film, and an insulating resin covering a surface of the electrode wiring pattern.

By such a structure, it is possible to reduce a distance between the electrodes by using the electrode wiring pattern formed on the base material resin film. As is apparent from Equation 2 which will be described below, therefore, an electrostatic capacitance Cₛ can be increased so that the excellent result of the measurement can be obtained.

In addition, the alcohol concentration detecting sensor is constituted by the base material resin film, the electrode wiring pattern formed on the base material resin film, and the insulating resin covering the surface of the electrode wiring pattern. Therefore, the sensor itself is flexible, thin, very small and compact, and can be installed everywhere so that the degree of freedom of a design can be increased.

Furthermore, the surface of the electrode wiring pattern is covered with the insulating resin. Therefore, an insulation between the electrodes is excellent and is not influenced by a moisture, and shielding can be carried out in order to prevent the influence of an electromagnetic wave generated from the body of a car or the like. Furthermore, an accurate measurement for the alcohol concentration can be executed.

Moreover, the electrode does not directly come in contact with the liquid to be inspected such as a gasoline. Therefore, a defective operation can be prevented from being caused by a deterioration with the passage of time, foreign matters in the gasoline or the like. Thus, it is possible to detect the alcohol concentration accurately and rapidly.

In addition, the alcohol concentration detecting apparatus according to the present invention is characterized in that the alcohol concentration detecting sensor body is stuck onto a substrate.

By such a structure, the alcohol concentration detecting sensor body is stuck onto the substrate. Therefore, it is easy to assemble and attach the alcohol concentration detecting sensor body into the apparatus.

Furthermore, the alcohol concentration detecting apparatus according to the present invention is characterized in that the electrode wiring pattern is obtained by selectively etching a conductive metallic foil laminated on one of surfaces of the base material resin film, thereby forming a wiring pattern taking a predetermines shape.

By such a structure, it is possible to obtain an electrode wiring pattern having a very small distance between the electrodes, for example, within a range of approximately 5 μm to 50 μm by etching. Therefore, the electrostatic capacitance Cₛ can be increased so that the excellent result of the measurement can be obtained.

In addition, the sensor itself is thin, very small and compact, and can be installed everywhere so that the degree of freedom of a design can be increased.

Moreover, the present invention provides an alcohol concentration detecting apparatus in which an alcohol concentration in the liquid to be inspected is detected by introducing a liquid to be inspected between electrodes of an alcohol concentration detecting sensor and by measuring a change in a specific inductive capacity of the liquid to be inspected between the electrodes with an oscillation frequency,
wherein the alcohol concentration detecting sensor comprises a substrate, an electrode wiring pattern formed on the substrate, and an insulating coat covering a surface of the electrode wiring pattern.

By such a structure, it is possible to reduce a distance between the electrodes by using the electrode wiring pattern formed on the substrate. As is apparent from the Equation 2 which will be described below, therefore, an electrostatic capacitance Cₛ can be increased so that the excellent result of the measurement can be obtained.

In addition, the alcohol concentration detecting sensor is constituted by the substrate, the electrode wiring pattern formed on the substrate, and the insulating coat covering the surface of the electrode wiring pattern. Therefore, the sensor itself is thin, very small and compact, and can be installed everywhere so that the degree of freedom of a design can be increased.

Furthermore, the surface of the electrode wiring pattern is covered with the insulating coat. Therefore, an insulation between the electrodes is excellent and is not influenced by a moisture, and shielding can be carried out in order to prevent the influence of an electromagnetic wave generated from the body of a car or the like. Furthermore, an accurate measurement for the alcohol concentration can be executed.

Moreover, the electrode does not directly come in contact with the liquid to be inspected such as a gasoline. Therefore, a defective operation can be prevented from being caused by a deterioration with the passage of time, foreign matters in the gasoline or the like. Thus, it is possible to detect the alcohol concentration accurately and rapidly.

In addition, the substrate is provided. Therefore, it is easy to assemble and attach the alcohol concentration detecting sensor into the apparatus.

Furthermore, the alcohol concentration detecting apparatus according to the present invention is characterized in that the electrode wiring pattern is obtained by selectively etching a conductive metallic thin film formed on one of surfaces of the substrate by sputtering, thereby forming a wiring pattern taking a predetermines shape.

By such a structure, it is possible to obtain an electrode wiring pattern having a thickness of 0.1 to 5 μm by sputtering at a very small distance between the electrodes, for example, within a range of approximately 5 μm to 50 μm by the sputtering. Therefore, the electrostatic capacitance Cₛ can be increased so that the excellent result of the measurement can be obtained.

In addition, the sensor itself is thin, very small and compact, and can be installed everywhere so that the degree of freedom of a design can be increased.

Moreover, the alcohol concentration detecting apparatus according to the present invention is characterized in that the insulating coat is formed by chemical vapor deposition (CVD).

By such a structure, it is possible to obtain, by the chemical vapor deposition (CVD), a very minute and thin insulating coat which is not influenced by the liquid to be inspected such as a gasoline or alcohol, for example, SiO₂, Al₂O₃ and the like. Thus, the sensor itself can be thin, very small and compact.

Furthermore, the alcohol concentration detecting apparatus according to the present invention is characterized in that the electrode wiring pattern has such a shape that positive and negative electrodes which are comb-toothed are alternately intricate.

By such a structure, the positive and negative electrodes which are comb-toothed are formed to be alternately intricate. Therefore, the electrodes having a very small distance therebetween can be provided to be compact as a whole.

Accordingly, it is possible to obtain an electrode wiring pattern having a very small distance between the electrodes, for example, within a range of approximately 5 μm to 50 μm by etching and sputtering, respectively. Therefore, the electrostatic capacitance Cₛ can be increased so that the excellent result of the measurement can be obtained.

In addition, the sensor itself is thinner, much smaller and more compact, and can be installed everywhere so that the degree of freedom of a design can be increased.

Moreover, the present invention provides an alcohol concentration detecting method of detecting an alcohol concentration in a liquid to be inspected by using the alcohol concentration detecting apparatus according to any of the aspects described above,
in which an alcohol concentration in the liquid to be inspected is detected by introducing a liquid to be inspected between electrodes of an alcohol concentration detecting sensor and by measuring a change in a specific inductive capacity of the liquid to be inspected between the electrodes with an oscillation frequency.

By such a structure, it is possible to reduce a distance between the electrodes by using the electrode wiring pattern formed on the base material resin film or the substrate. As is apparent from the Equation 2 which will be described below, therefore, the electrostatic capacitance Cₛ can be increased so that the excellent result of the measurement can be obtained.

In addition, the alcohol concentration detecting sensor itself is thin, very small and compact, and can be installed everywhere so that the degree of freedom of a design can be increased.

Furthermore, the surface of the electrode wiring pattern is covered with the insulating resin or the insulating coat. Therefore, an insulation between the electrodes is excellent and is not influenced by a moisture, and shielding can be carried out in order to prevent the influence of an electromagnetic wave generated from the body of a car or the like. Furthermore, an accurate measurement for the alcohol concentration can be executed.

Moreover, the electrode does not directly come in contact with the liquid to be inspected such as a gasoline. Therefore, a defective operation can be prevented from being caused by a deterioration with the passage of time, foreign matters in the gasoline or the like. Thus, it is possible to detect the alcohol concentration accurately and rapidly.

In addition, the alcohol concentration detecting method according to the present invention is characterized in that the liquid to be inspected is a gasoline containing alcohol.

By such a structure, the alcohol concentration in the gasoline can be detected accurately and rapidly, and it is possible to control a torque to be constant by excessively adding a gasoline corresponding to the amount of addition of alcohol, for example, ethanol as an antiknocking agent.

Furthermore, the present invention provides a method of manufacturing an alcohol concentration detecting sensor, comprising:
a conductive metallic foil sticking step of sticking a conductive metallic foil onto one of surfaces of abase material resin film;
a photoresist applying step of applying a photoresist onto a whole upper surface of the conductive metallic foil;
a photoresist exposing step of exposing the photoresist to take a desirable electrode wiring pattern shape by using a photoresist mask;
a photoresist dissolving and removing step of dissolving and removing the exposed photoresist portion with a developing solution;
an etching step of etching and removing a conductive metallic foil portion which is not covered with the photoresist, with an etchant;
a photoresist dissolving and removing step of dissolving and removing the photoresist; and
an insulating resin applying step of applying an insulating resin onto the surface from which the photoresist is removed, thereby obtaining an alcohol concentration detecting sensor body.

By such a structure, it is possible to obtain an electrode wiring pattern having a very small distance between the electrodes, for example, within a range of approximately 5 μm to 50 μm. Consequently, it is possible to easily supply, on a large scale, an alcohol concentration detecting sensor in which the electrostatic capacitance Cₛ can be increased so that the excellent result of the measurement can be obtained. Furthermore, the sensor itself is thin, very small and compact, and can be installed everywhere so that the degree of freedom of a design can be increased.

In addition, the alcohol concentration detecting sensor manufacturing method according to the present invention comprising a substrate sticking step of sticking the alcohol concentration detecting sensor body obtained at the insulating resin applying step onto a substrate.

By such a structure, the alcohol concentration detecting sensor body is stuck onto the substrate. Consequently, it is possible to easily supply, on a large scale, an alcohol concentration detecting sensor in which the alcohol concentration detecting sensor body can easily be assembled and attached into the apparatus.

Furthermore, the alcohol concentration detecting sensor manufacturing method according to the present invention is characterized in that the base material resin film is a polyimide resin film.

By such a structure, the electrode wiring pattern can be formed on the flexible, thin and small polyimide resin film. Consequently, it is possible to easily supply, on a large scale, an alcohol concentration detecting sensor in which the sensor itself is thin, very small and compact, and can be installed everywhere and the degree of freedom of a design can be increased.

Moreover, the alcohol concentration detecting sensor manufacturing method according to the present invention is characterized in that the conductive metallic foil is a copper foil.

By such a structure, the electrode wiring pattern can be formed by the copper foil. Consequently, it is possible to easily supply, on a large scale, an alcohol concentration detecting sensor in which a high conductivity can be obtained and an alcohol concentration can be detected very accurately and rapidly.

In addition, the alcohol concentration detecting sensor manufacturing method according to the present invention is characterized in that the insulating resin is constituted by at least one selected from an urethane resin, a polyimide resin and an epoxy type resin.

By using such a resin as the insulating resin, it is possible to easily apply the insulating resin onto the surface of the electrode wiring pattern.

Moreover, the present invention provides a method of manufacturing an alcohol concentration detecting sensor, comprising:
a conductive metallic thin film forming step of forming a conductive metallic thin film on one of surfaces of a substrate by sputtering;
a photoresist applying step of applying a photoresist onto a whole upper surface of the conductive metallic thin film;
a photoresist exposing step of exposing the photoresist to take a desirable electrode wiring pattern shape by using a photoresist mask;
a photoresist dissolving and removing step of dissolving and removing the exposed photoresist portion with a developing solution;
an etching step of dry etching and removing a conductive metallic thin film portion which is not covered with the photoresist;
a photoresist dissolving and removing step of dissolving and removing the photoresist; and
an insulating coat forming step of forming an insulating coat on a surface of the electrode wiring pattern from which the photoresist is removed, by chemical vapor deposition (CVD).

By such a structure, it is possible to obtain an electrode wiring pattern having a thickness of 0.1 to 5 μm by sputtering at a very small distance between the electrodes, for example, within a range of approximately 5 μm to 50 μm. Therefore, it is possible to easily supply, on a large scale, an alcohol concentration detecting sensor in which the electrostatic capacitance Cₛ can be increased so that the excellent result of the measurement can be obtained. Furthermore, the sensor itself is thin, very small and compact, and can be installed everywhere so that the degree of freedom of a design can be increased.

Moreover, the alcohol concentration detecting sensor manufacturing method according to the present invention is characterized in that the substrate is constituted by at least one selected from ceramics, glass and a resin substrate.

By such a structure, a conductive metallic thin film constituting the electrode wiring pattern can easily be formed, through sputtering, on the substrate constituted by such a material, and furthermore, the substrate formed by the same material is provided. Consequently, it is possible to easily supply, on a large scale, an alcohol concentration detecting sensor which can easily be assembled and attached into the apparatus.

Furthermore, the alcohol concentration detecting sensor manufacturing method according to the present invention is characterized in that the conductive metallic thin film is constituted by at least one selected from platinum, nickel, copper and titanium.

By such a structure, it is possible to easily form the conductive metallic thin film constituting the electrode wiring pattern on the substrate by sputtering.

In addition, the alcohol concentration detecting sensor manufacturing method according to the present invention is characterized in that the insulating coat is constituted by at least one minute insulating coat selected from SiO₂, Al₂O₃ and the like.

By such a structure, it is possible to easily form the insulating coat on the surface of the electrode wiring pattern by chemical vapor deposition (CVD).

Moreover, the alcohol concentration detecting sensor manufacturing method according to the present invention is characterized in that the electrode wiring pattern has such a shape that positive and negative electrodes which are comb-toothed are alternately intricate.

By such a structure, the positive and negative electrodes which are comb-toothed are formed to be alternately intricate. Therefore, it is possible to easily supply, on a large scale, an alcohol concentration detecting sensor in which the electrodes having a very small distance therebetween can be provided to be compact as a whole, the sensor itself is thin, very small and compact, and can be installed everywhere so that the degree of freedom of a design can be increased.

### BRIEF DESCRIPTION OF THE DRAWINGS

FIG. 1 is a schematic top view showing an example of an alcohol concentration detecting apparatus according to the present invention.

FIG. 2 is a sectional view taken along an A-A line in FIG. 1.

FIG. 3 is a right side view of FIG. 1.

FIG. 4 is a left side view of FIG. 1.

FIG. 5 is a graph showing a relationship between the concentration of alcohol and an electrostatic capacitance FIG. 6 is a schematic circuit diagram showing the structure of the alcohol concentration detecting apparatus according to the present invention.

FIG. 7 is a schematic diagram showing a square-wave voltage to be applied by the alcohol concentration detecting apparatus according to the present invention.

FIG. 8 is a graph showing a relationship between an alcohol concentration and an oscillation frequency.

FIG. 9 is a schematic perspective view showing an example of an alcohol concentration detecting sensor in the alcohol concentration detecting apparatus according to the present invention.

FIG. 10 is a schematic top view showing an electrode wiring pattern in FIG. 9.

FIG. 11 is an enlarged view showing a B portion in FIG. 10.

FIG. 12 is a partially enlarged sectional view taken along a C-C line in FIG. 9.

FIG. 13 is a schematic view showing a method of manufacturing the alcohol concentration detecting sensor in the alcohol concentration detecting apparatus according to the present invention.

FIG. 14 is a schematic perspective view showing another example of the alcohol concentration detecting sensor in the alcohol concentration detecting apparatus according to the present invention.

FIG. 15 is a schematic top view showing an electrode wiring pattern in FIG. 14.

FIG. 16 is an enlarged view showing a B portion in FIG. 15.

FIG. 17 is a partially enlarged sectional view taken along a C-C line in FIG. 14.

FIG. 18 is a schematic view showing a method of manufacturing the alcohol concentration detecting sensor in the alcohol concentration detecting apparatus according to the present invention.

FIG. 19 is a schematic view showing a conventional optical alcohol concentration measuring apparatus.

FIG. 20 is a sectional view showing a conventional electrostatic capacitance type alcohol concentration sensor.

### BEST MODE FOR CARRYING OUT THE INVENTION

An embodiment (example) of the present invention will be described below in more detail with reference to the drawings.

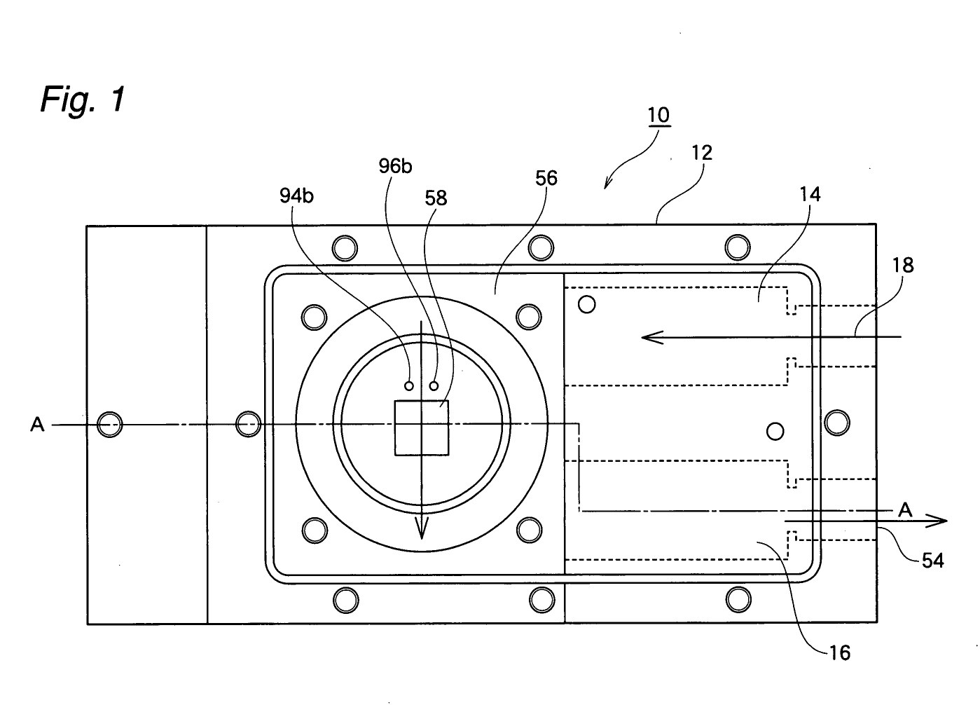

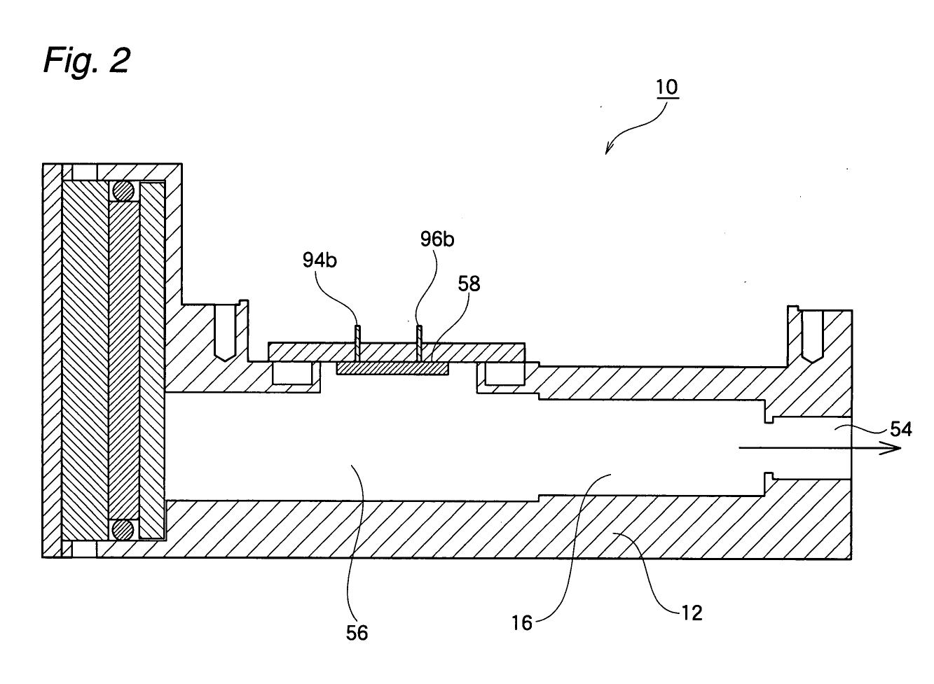

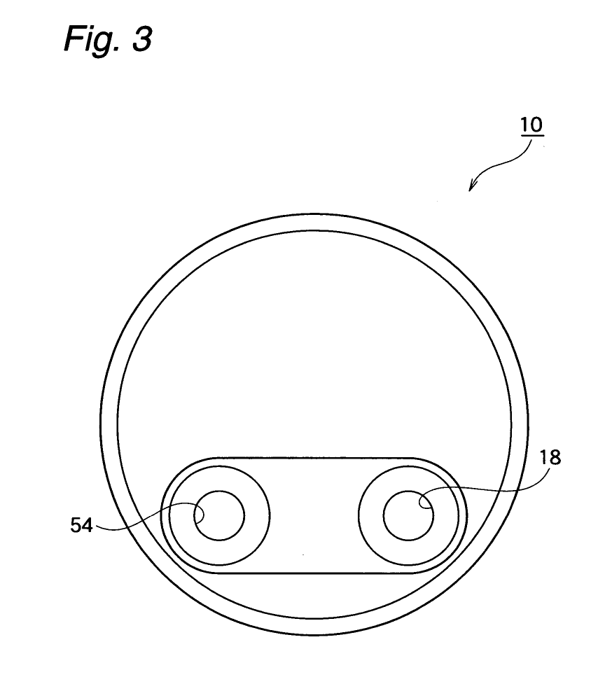

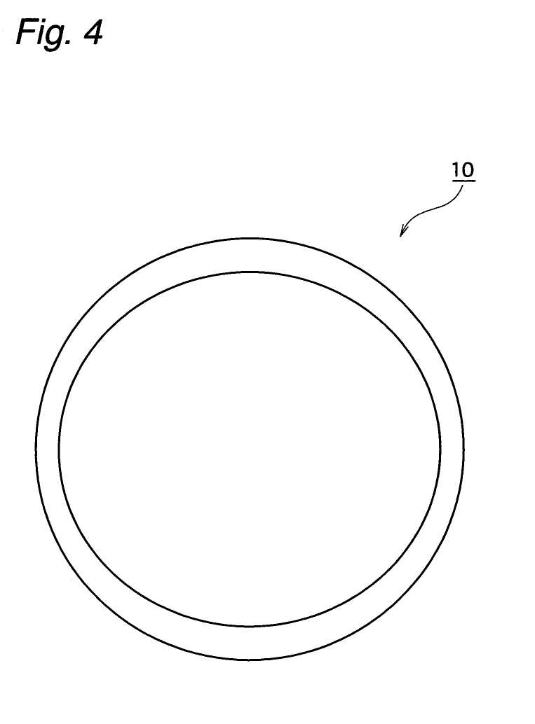

FIG. 1 is a schematic top view showing an example of an alcohol concentration detecting apparatus according to the present invention, FIG. 2 is a sectional view taken along an A-A line in FIG. 1, FIG. 3 is a right side view of FIG. 1, and FIG. 4 is a left side view of FIG. 1.

As shown in FIGS. 1 to 4, an alcohol concentration detecting apparatus 10 according to the present invention comprises an alcohol concentration detecting apparatus body 12, and a first passage 14 and a second passage 16 which are formed in the alcohol concentration detecting apparatus body 12.

As shown in an arrow of FIG. 1, a liquid to be inspected which flows from an inspected liquid inlet port 18 into the first passage 14 passes through an alcohol content detecting chamber 56.

On the other hand, in a state in which the liquid to be inspected which flows into the first passage 14 through the inspected liquid inlet port 18 then stays temporarily in the alcoholic content detecting chamber 56, the concentration of the alcohol content is detected by an alcohol concentration detecting sensor 58 if the liquid to be inspected contains alcohol. Then, the same liquid is then discharged from the alcohol content detecting chamber 56 through an inspected liquid discharge port 54 of the second passage 16.

In the alcohol concentration detecting sensor 58, a difference in an electrostatic capacitance is utilized depending on a difference between the specific inductive capacity of alcohol contained in the liquid to be inspected and the specific inductive capacity of the liquid to be inspected based on the following Equation 2.
Cₛ=ε₀(S/D)(εra(α/100)+εrb(1−α/100)) Equation 2

Herein, S represents an opposed area of an electrode, D represents a distance between the electrodes, ε₀ represents a specific inductive capacity of a vacuum (8.854E-12F/m), εra represents a specific inductive capacity of alcohol, εrb represents a specific inductive capacity of a liquid to be inspected, and α represents an alcohol concentration (%).

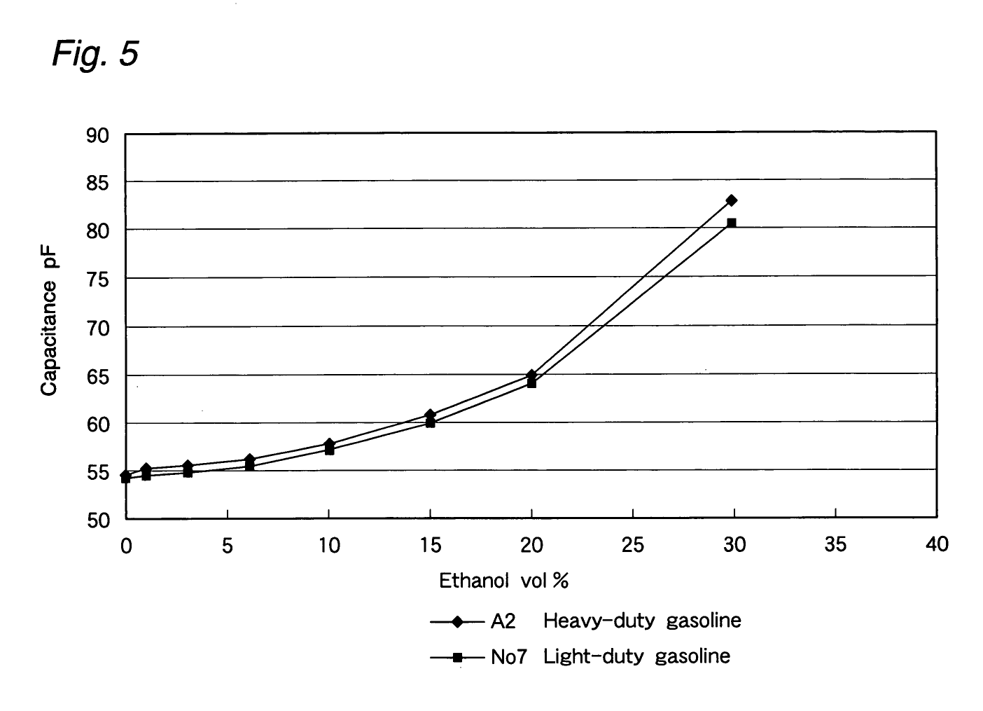

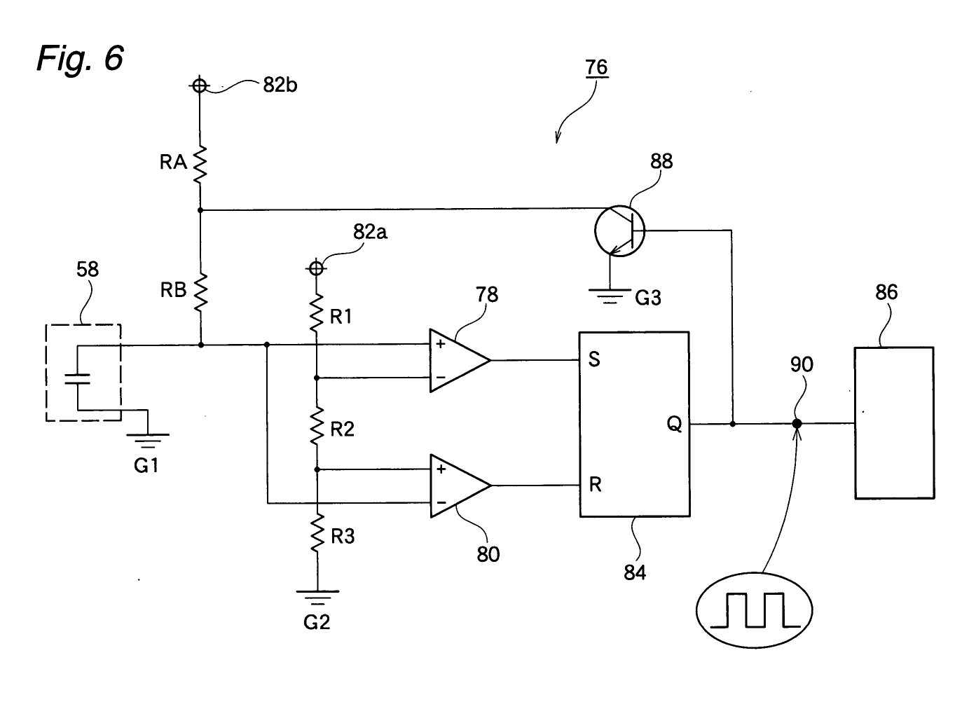

More specifically, as shown in a graph of FIG. 5 which represents a relationship between an alcohol concentration and an electrostatic capacitance, the alcohol concentration and the electrostatic capacitance have a correlation, and the alcohol concentration is detected by utilizing the correlation.

FIG. 5 shows an example in which ethanol is used for the alcohol and a gasoline is used for the liquid to be inspected.

Moreover, the alcohol concentration detecting apparatus 10 according to the present invention which uses the alcohol concentration detecting sensor 58 comprises a detecting control portion 76 having a structure shown in a diagram of FIG. 6 illustrating the schematic structure of a circuit.

As shown in FIG. 6, in the detecting control portion 76, one of the electrodes of the alcohol concentration detecting sensor 58 is grounded G1. In addition, the other electrode of the alcohol concentration detecting sensor 58 branches to be connected to the positive and negative inputs of amplifiers (operational amplifiers) 78 and 80.

Moreover, resistors R1 to R3 are connected to a negative 82a of a power supply 82, and furthermore, the negative input of the amplifier 78 is connected between R1 and R2. Furthermore, the positive input of the amplifier 80 is connected between R2 and R3, and an end of R3 is grounded G2.

The outputs of the amplifiers 78 and 80 are connected to S and R inputs of a flip-flop circuit 84, respectively. The output of the flip-flop circuit 84 is input to the frequency counter of a computer 86.

Furthermore, the wiring of one of the electrodes of the alcohol concentration detecting sensor 58 branches and is connected to a positive 82b of the power supply 82 through resistors RA and RB. A transistor 88 is connected between the resistors RA and RB, and the output of the transistor is connected between the output of the flip-flop circuit 84 and the computer 86. G3 denotes a ground of the transistor 88.

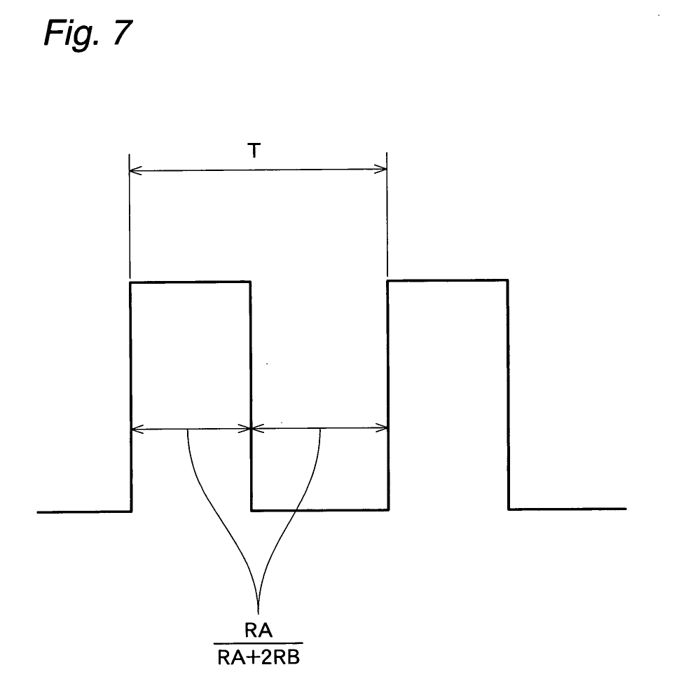

In the detecting control portion 76 having such a structure, a square-wave voltage shown in FIGS. 6 and 7 is applied at 90 in FIG. 6.

As is expressed in the following Equation 3, consequently, a relationship between an oscillation frequency f and an electrostatic capacitance Cₛ is obtained.
1/T=f=RA/(RA+2RB)·1/Cs(Hz) Equation 3

In this case, it is possible to determine an amplitude T by properly setting a duty ratio/(RA+2RB). In the present example, 1.44 was employed for the duty ratio.

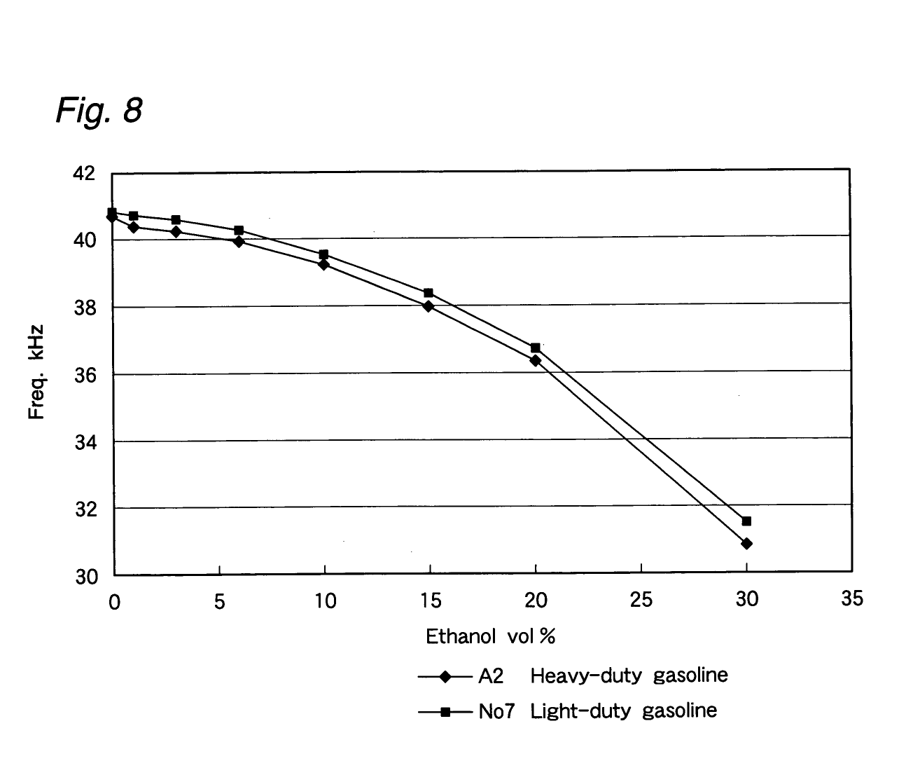

From such a relationship, a correlation is taken based on the graph of FIG. 5. As shown in a graph of FIG. 8 representing a relationship between an alcohol concentration and an oscillation frequency, consequently, it is apparent that the alcohol concentration and the oscillation frequency have a correlation. Thus, it is possible to detect the alcohol concentration.

The data in FIGS. 5 and 8 are previously stored in the storage portion of the computer and are compared with data obtained in the detecting control portion 76 so that the alcohol concentration can be detected.

As is apparent from the Equation 2, in order to obtain the excellent result of a measurement, it is preferable that the distance D between electrodes should be reduced to increase the electrostatic capacitance Cₛ.

In the alcohol concentration detecting apparatus 10 according to the present invention, therefore, the alcohol concentration detecting sensor 58 is constituted in the following manner.

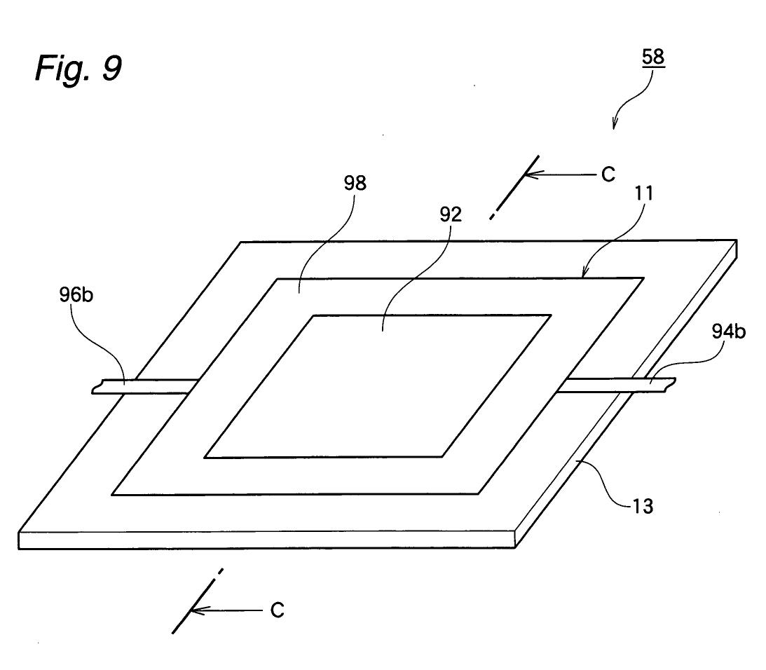

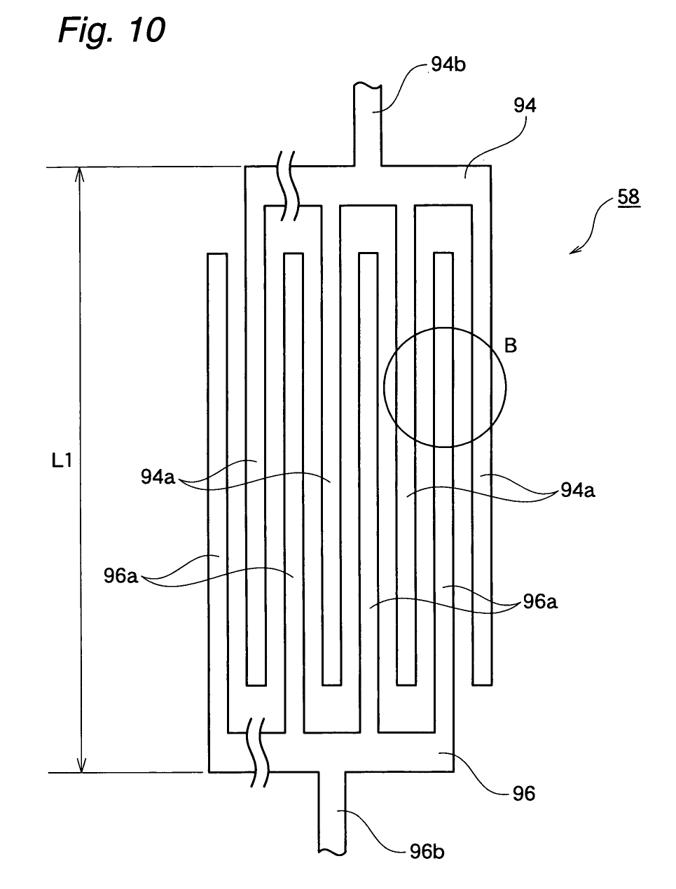

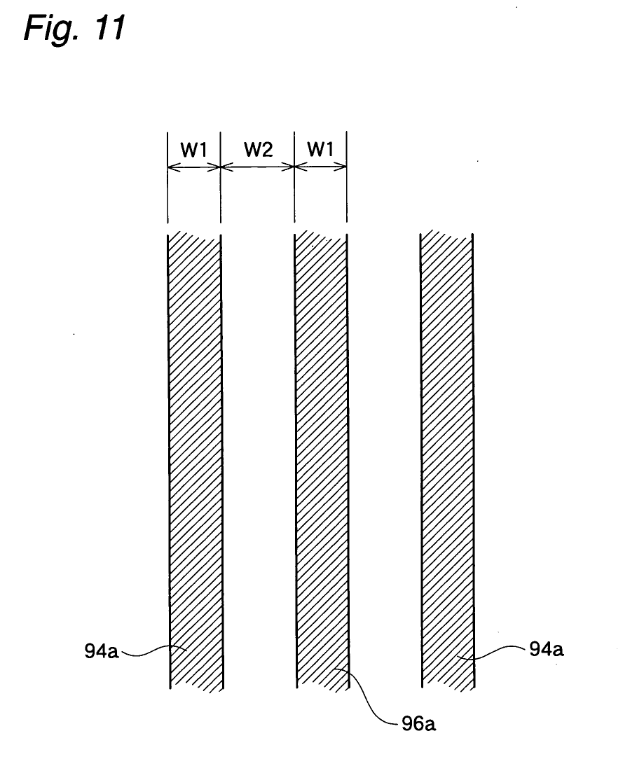

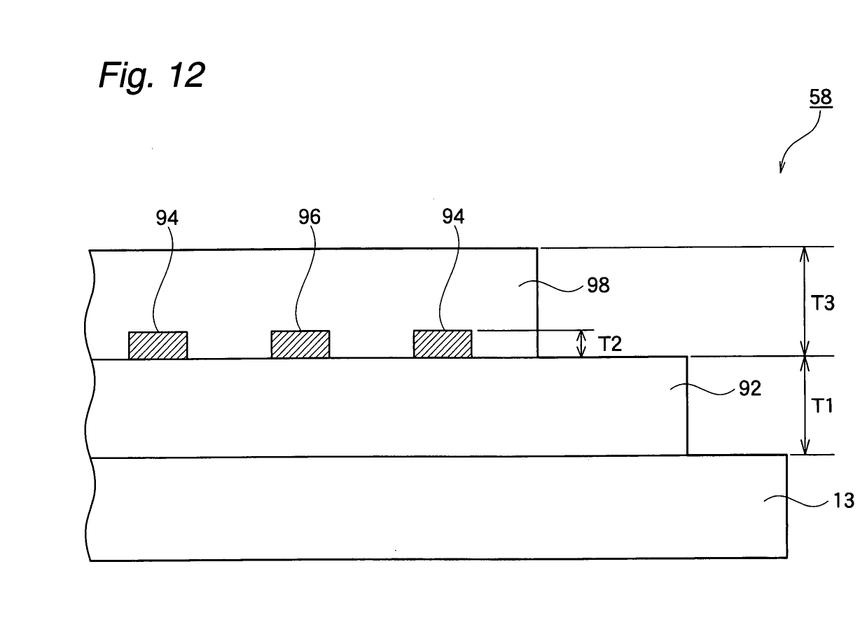

More specifically, FIG. 9 is a schematic perspective view showing an example of the alcohol concentration detecting sensor 58 of the alcohol concentration detecting apparatus according to the present invention, FIG. 10 is a schematic top view showing an electrode wiring pattern in FIG. 9, FIG. 11 is an enlarged view showing a B portion in FIG. 10, and FIG. 12 is a partially enlarged sectional view taken along a C-C line in FIG. 9.

As shown in FIGS. 9 to 12, the alcohol concentration detecting sensor 58 includes an alcohol concentration detecting sensor body 11 constituted by a base material resin film 92, electrode wiring patterns 94 and 96 formed on the base material resin film 92, and an insulating resin 98 covering the surfaces of the electrode wiring patterns 94 and 96. The alcohol concentration detecting sensor body 11 is stuck to a substrate 13 with an adhesive which is not shown.

In this case, it is preferable that a polyimide resin film should be used for the base material resin film 92 in consideration of a flexibility, a chemical resistance and the like. As shown in FIG. 12, moreover, a thickness T1 is not particularly restricted.

Furthermore, the electrode wiring pattern 94 on a positive side and the electrode wiring pattern 96 on a ground (a negative side) have such shapes that positive electrodes 94a and negative electrodes 96a which are comb-toothed are alternately intricate, respectively. In FIG. 9, 94b and 96b denote a fetch electrode portion, respectively.

By such a structure, a plurality of electrodes having a very small distance therebetween can be provided to be compact as a whole.

In this case, while a length L1 of the electrode is not particularly restricted as shown in FIG. 10, it is desirably set to be 100 μm or more in consideration of the electrostatic capacitance of the liquid to be inspected. In the present example, a length of 10 mm was employed for L1.

While a width W1 of each of the positive electrode 94a and the negative electrode 96a is not particularly restricted as shown in FIG. 11, moreover, in consideration of the electrostatic capacitance, it is preferably set to be 1 to 50 μm, and more preferably, 5 to 15 μm. Furthermore, a width W2 between the positive electrode 94a and the negative electrode 96a is not particularly restricted but is preferably set to be 1 to 50 μm, and more preferably 5 to 15 μm in consideration of the electrostatic capacitance. In the present example, W1/W2=30/30 μm was employed.

While the numbers of the positive electrodes 94a and the negative electrodes 96a which are comb-toothed are not particularly restricted, moreover, in consideration of the electrostatic capacitance, they are preferably equal to or larger than 1, and more preferably, are more increased. In the present example, 64 pairs (128 in total) comb-toothed electrodes were used.

While a thickness T2 of each of the electrode wiring patterns 94 and 96 is not particularly restricted as shown in FIG. 12, furthermore, in consideration of the electrostatic capacitance, it is preferably set to be 1 to 50 μm, and more preferably, 5 to 15 μm. In the present example, T2 of 10 μm was employed.

In this case, the electrode wiring patterns 94 and 96 are obtained by selectively etching a conductive metallic foil laminated on one of the surfaces of the base material resin film 92 to form wiring patterns taking predetermined shapes as will be described below.

Although such a conductive metallic foil is not particularly restricted, a copper foil is preferable. Consequently, a high conductivity can be obtained and the concentration of alcohol can be detected very accurately and rapidly.

Furthermore, it is preferable that the insulating resin 98 should be formed by at least one insulating resin selected from an urethane resin, a polyimide resin and an epoxy type resin.

By using such a resin as the insulating resin 98, it is possible to easily apply the insulating resin onto the surfaces of the electrode wiring patterns 94 and 96.

While a thickness T3 of the insulating resin 98 is not particularly restricted as shown in FIG. 12, moreover, in consideration of the fact that the electrostatic capacitance of the insulating resin itself does not influence sensing, it is desirable that the thickness T3 should be smaller with an insulating property and a strength maintained. In the present example, T3 of 18 μm was employed.

While the material of the substrate 13 is not particularly restricted, furthermore, in consideration of a specific inductive capacity, it is possible to employ a glass substrate, a ceramics substrate, a resin substrate or the like. Although the thickness is not particularly restricted, it is preferably set to be 100 to 1000 μm, and more preferably, 250 to 600 μm in consideration of an insulating property, a strength and the like. In the present example, a thickness of 360 μm was employed.

By such a structure, it is possible to reduce the distance between the electrodes by using the electrode wiring patterns 94 and 96 formed on the base material resin film 92. As is apparent from the Equation 2, therefore, the electrostatic capacitance Cₛ can be increased so that the excellent result of the measurement can be obtained.

In addition, the alcohol concentration detecting sensor 58 is constituted by the base material resin film 92, the electrode wiring patterns 94 and 96 formed on the base material resin film 92, and the insulating resin 98 covering the surfaces of the electrode wiring patterns 94 and 96. For this reason, the sensor itself is flexible, thin, very small and compact, and can be installed everywhere so that the degree of freedom of a design can be enhanced.

Since the surfaces of the electrode wiring patterns 94 and 96 are covered with the insulating resin 98, furthermore, an insulation between the electrodes can be enhanced and is not influenced by a moisture, and shielding can be carried out so as not to be influenced by an electromagnetic wave generated from a body of a car or the like. Furthermore, the alcohol concentration can be measured accurately.

Moreover, the electrode does not directly come in contact with the liquid to be inspected such as a gasoline. Therefore, a defective operation can be prevented from being caused by a deterioration with passage of time, foreign matters in the gasoline or the like. Thus, it is possible to detect the alcohol concentration accurately and rapidly.

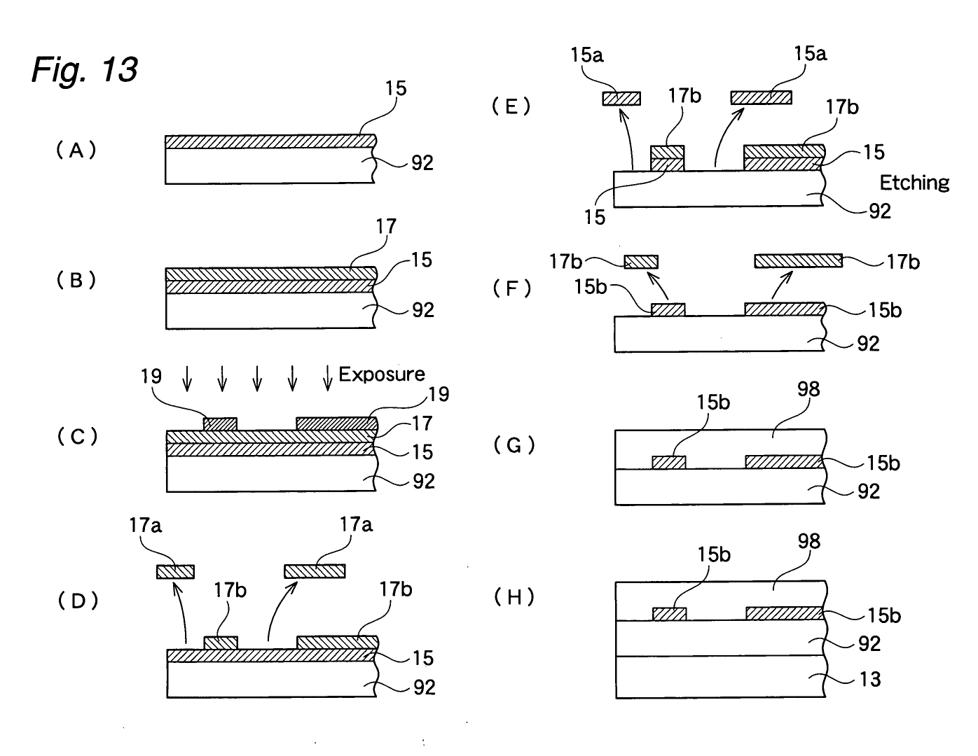

With reference to FIG. 13, description will be given to a method of manufacturing the alcohol concentration detecting sensor of the alcohol concentration detecting apparatus according to the present invention which has such a structure.

As shown in FIG. 13(A), first of all, a conductive metallic foil 15 is stuck to one of the surfaces of the base material resin film 92 by contact bonding with an adhesive which is not shown (a conductive metallic foil sticking step).

As shown in FIG. 13(B), then, a photoresist 17 is applied onto the whole upper surface of the conductive metallic foil 15 by using a spin coater (3000 rpm), for example (a photoresist applying step).

As shown in FIG. 13(C), next, the photoresist 17 is exposed by ultraviolet rays to take a desirable electrode wiring pattern shape by using a photoresist mask 19 taking a shape corresponding to a predetermined wiring pattern, for example (a photoresist exposing step).

As shown in FIG. 13(D), then, a photoresist portion 17a thus exposed is dissolved and removed with a developing solution (a photoresist dissolving and removing step).

As shown in FIG. 13(E), thereafter, a conductive metallic foil portion 15a which is not covered with the photoresist 17b is subjected to an etching treatment with an etchant such as acid or alkali and is thus removed to obtain a predetermined wiring pattern shape 15b (an etching step).

As shown in FIG. 13(F), subsequently, the photoresist 17b is dissolved and removed with a dissolving and removing solution such as acetone (a photoresist dissolving and removing step).

As shown in FIG. 13(G), next, the insulating resin 98 is applied onto the surface from which the photoresist is removed by screen printing, for example, and the alcohol concentration detecting sensor body 11 is thus obtained (an insulating resin applying step).

As shown in FIG. 13(H) and FIG. 12, finally, the alcohol concentration detecting sensor body 11 obtained at the insulating resin applying step is stuck onto the substrate 13 (a substrate sticking step).

According to the method of manufacturing the alcohol concentration detecting sensor 58 of the alcohol concentration detecting apparatus according to the present invention, it is possible to obtain an electrode wiring pattern in which a distance between electrodes is very small, for example, approximately 5 μm to 50 μm. Therefore, it is possible to easily supply, on a large scale, an alcohol concentration detecting sensor in which the electrostatic capacitance Cₛ can be increased and the excellent result of the measurement can be obtained. Furthermore, the sensor itself is thin, very small and compact, and can be installed everywhere so that the degree of freedom of a design can be enhanced.

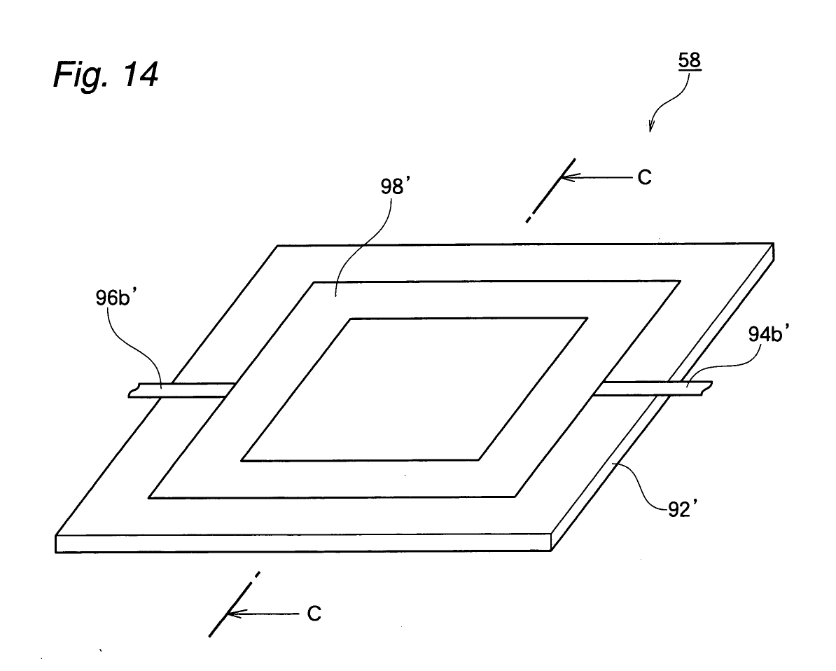

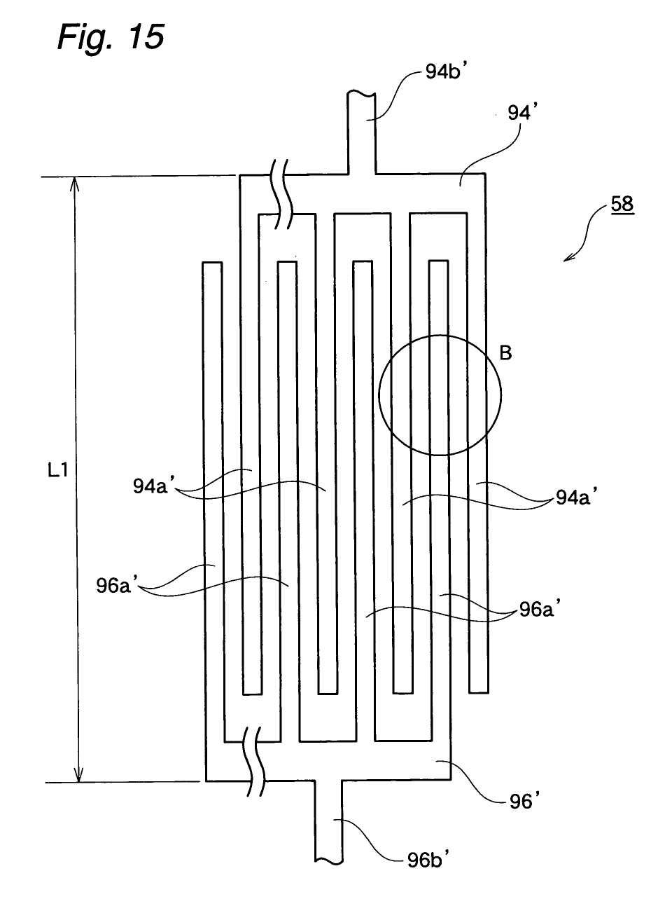

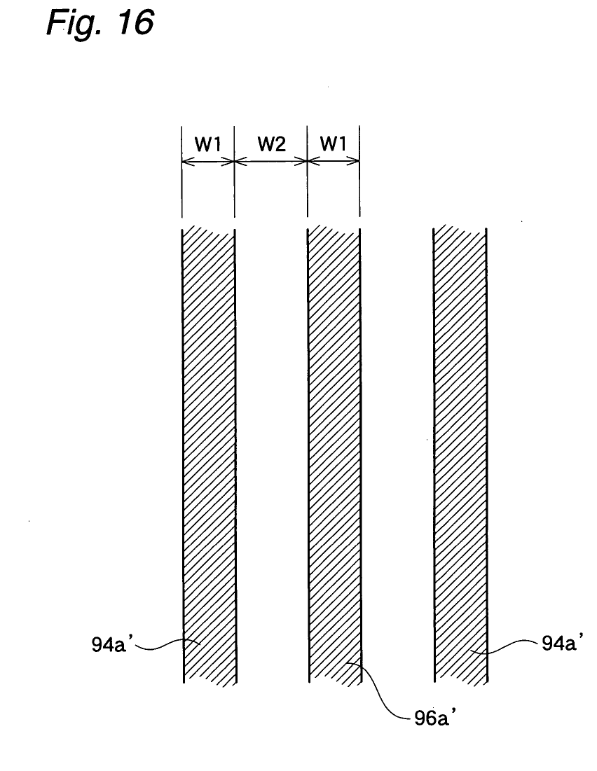

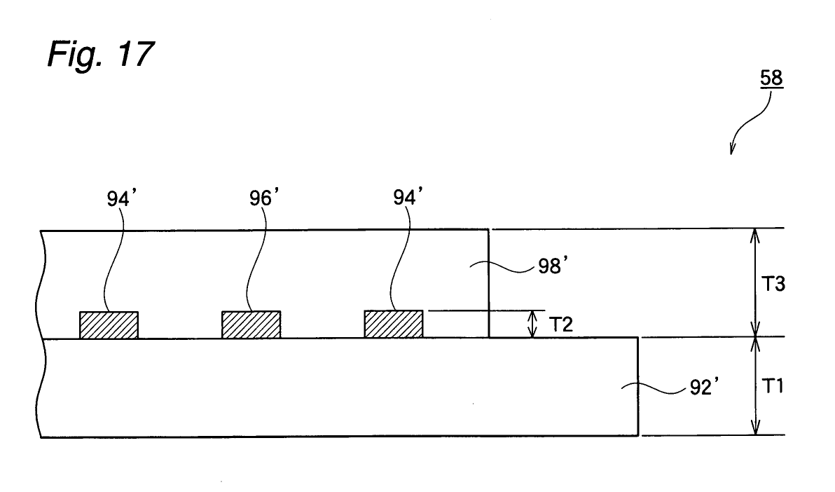

FIG. 14 is a schematic perspective view showing another example of the alcohol concentration detecting sensor 58 of the alcohol concentration detecting apparatus according to the present invention, FIG. 15 is a schematic top view showing an electrode wiring pattern in FIG. 14, FIG. 16 is an enlarged view showing a B portion in FIG. 15, and FIG. 17 is a partially enlarged sectional view taken along a C-C line in FIG. 14.

The alcohol concentration detecting sensor 58 according to the present example has basically the same structure as that of the alcohol concentration detecting sensor 58 according to the example shown in FIGS. 9 to 12. Therefore, the same components are indicated as the reference numerals having a dash and detailed description thereof will be omitted.

The alcohol concentration detecting sensor 58 according to the present example comprises a substrate 92′, electrode wiring patterns 94′ and 96′ formed on the substrate 92′, and an insulating coat 98′ covering the surfaces of the electrode wiring patterns 94′ and 96′.

In this case, the electrode wiring patterns 94′ and 96′ are obtained by selectively etching a conductive metallic thin film formed on one of the surfaces of the substrate 92′ through sputtering to provide a wiring pattern taking a predetermined shape.

While such a conductive metallic thin film is not particularly restricted, nickel, copper, platinum or the like can be used and the platinum is desirably used in consideration of an oxidation resistance or the like.

While a thickness T2 of each of the electrode wiring patterns 94′ and 96′ is not particularly restricted as shown in FIG. 17, moreover, in consideration of an efficiency in the formation of a thin film by the sputtering, it is preferably set to be 0.1 to 1.0 μm, and more preferably, 0.1 to 0.5 μm.

While the material of the substrate 92′ is not particularly restricted, furthermore, in consideration of the fact that the material is not influenced by sputtering or the like, a glass substrate, a ceramics substrate such as alumina, a resin substrate or the like can be employed. While the thickness is not particularly restricted, it is preferably set to be 100 to 1000 μm, and more preferably, 250 to 600 μm in consideration of an insulating property, a strength or the like. In the present example, a thickness of 360 μm was used. A size depends on the size of a sputtering device, and desirably, 2-inch and 4-inch square sizes can be used.

While the insulating coat 98′ is not particularly restricted, moreover, it is preferably constituted by at least one minute insulating coat selected from SiO₂, Al₂O₃ and the like.

In this case, it is preferable that the insulating coat 98′ should be formed by chemical vapor deposition (CVD).

By such a structure, it is possible to obtain, by the chemical vapor deposition (CVD), a very minute and thin insulating coat which is not influenced by a liquid to be inspected such as a gasoline or alcohol, for example, SiO₂, Al₂O₃ or the like, and the sensor itself can be thin, very small and compact.

While a thickness T3 of the insulating resin 98′ is not particularly restricted as shown in FIG. 17, moreover, in consideration of the fact that the electrostatic capacitance of the insulating coat itself such as an insulating property, a strength and the like does not influence sensing, it is desirable that the thickness T3 should be smaller with an insulating property and a strength maintained. In the present example, T3 of 1 μm was employed.

By such a structure, it is possible to reduce the distance between the electrodes by using the electrode wiring patterns 94′ and 96′ formed on the substrate 92′. As is apparent from the Equation 2, therefore, the electrostatic capacitance Cₛ can be increased and the excellent result of the measurement can be obtained.

In addition, the alcohol concentration detecting sensor is constituted by the substrate 92′, the electrode wiring patterns 94′ and 96′ formed on the substrate 92′, and the insulating coat 98′ covering the surfaces of the electrode wiring patterns 94′ and 96′. For this reason, the sensor itself is thin, very small and compact, and can be installed everywhere so that the degree of freedom of a design can be enhanced.

Since the surfaces of the electrode wiring patterns are covered with the insulating coat 98′, furthermore, an insulation between the electrodes can be enhanced and is not influenced by a moisture, and shielding can be carried out so as not to be influenced by an electromagnetic wave generated from a body of a car or the like. Furthermore, the alcohol concentration can be measured accurately.

Moreover, the electrode does not directly come in contact with the liquid to be inspected such as a gasoline. Therefore, a defective operation can be prevented from being caused by a deterioration with passage of time, foreign matters in the gasoline or the like. Thus, it is possible to detect the alcohol concentration accurately and rapidly.

In addition, the substrate 92′ is provided. Therefore, it is easy to assemble and attach the alcohol concentration detecting sensor into the apparatus.

Furthermore, it is possible to obtain an electrode wiring pattern having a thickness of 0.1 to 5 μm by sputtering at a very small distance between the electrodes, for example, within a range of approximately 5 μm to 50 μm by the sputtering. Therefore, the electrostatic capacitance Cₛ can be increased so that the excellent result of the measurement can be obtained.

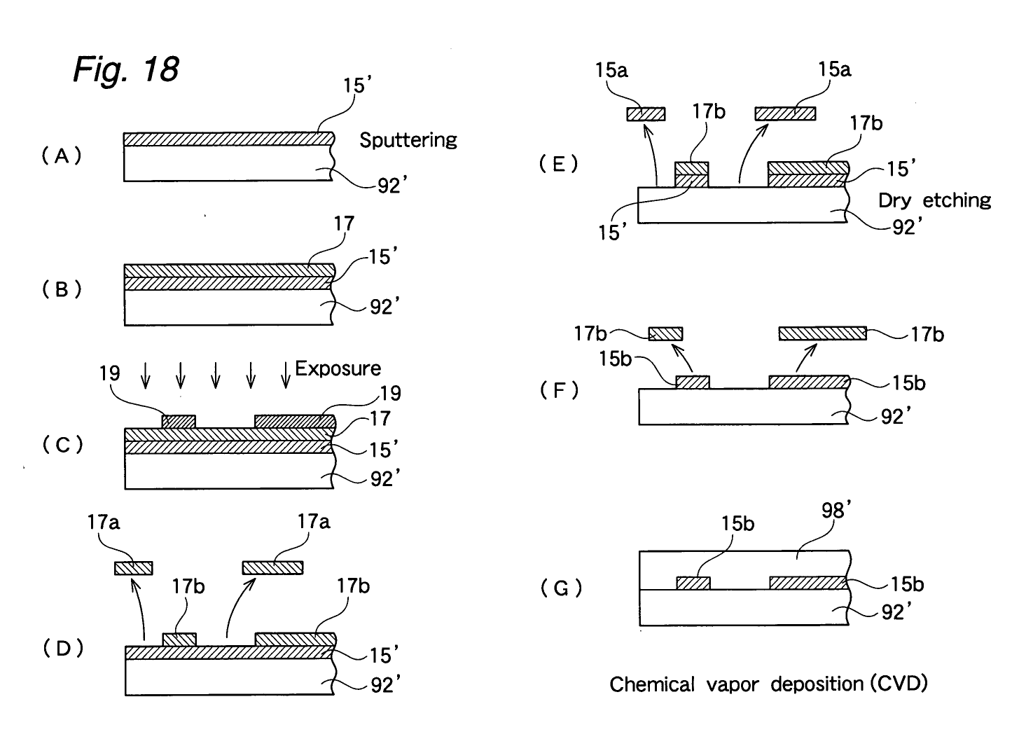

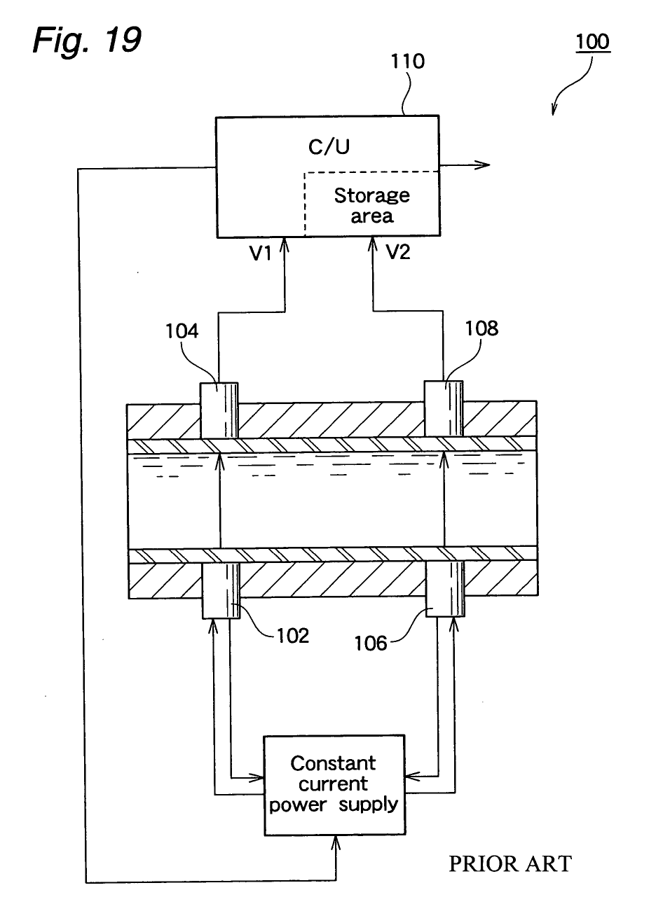

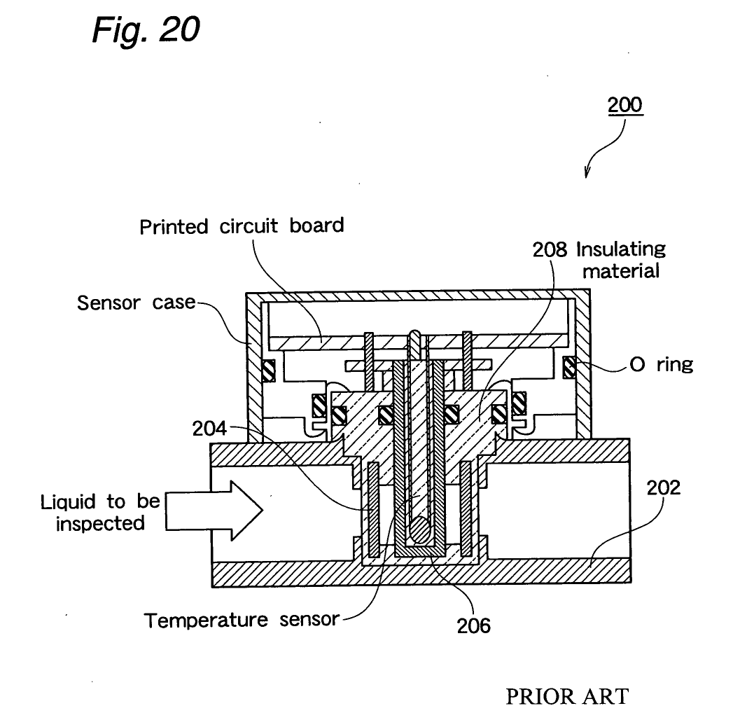

With reference to FIG. 18, description will be given to a method of manufacturing the alcohol concentration detecting sensor of the alcohol concentration detecting apparatus according to the present invention which has such a structure.

As shown in FIG. 18(A), first of all, a conductive metallic thin film 15′ is formed on one of the surfaces of the substrate 92′ by the sputtering (a conductive metallic thin film forming step).

As shown in FIG. 18(B), then, a photoresist 17 is applied onto the whole upper surface of the conductive metallic thin film 15′ by using a spin coater (3000 rpm), for example (a photoresist applying step).

As shown in FIG. 18(C), next, the photoresist 17 is exposed by ultraviolet rays to take a desirable electrode wiring pattern shape by using a photoresist mask 19 taking a shape corresponding to a predetermined wiring pattern, for example (a photoresist exposing step).

As shown in FIG. 18(D), then, a photoresist portion 17a thus exposed is dissolved and removed with a developing solution (a photoresist dissolving and removing step).

As shown in FIG. 18(E), thereafter, a conductive metallic thin film portion 15a which is not covered with the photoresist 17b is subjected to a dry etching treatment by using an argon ion or the like, for example, and is thus removed to obtain a predetermined wiring pattern shape 15b (an etching step).

As shown in FIG. 18(F), subsequently, the photoresist 17b is dissolved and removed with a dissolving and removing solution such as acetone (a photoresist dissolving and removing step).

As shown in FIG. 18(G), finally, the insulating coat 98′ is formed, by the chemical vapor deposition (CVD), on the surface from which the photoresist is removed (an insulating coat forming step).

By such a structure, it is possible to obtain an electrode wiring pattern having a thickness of 0.1 to 5 μm by the sputtering at a very small distance between the electrodes, for example, within a range of approximately 5 μm to 50 μm. Therefore, it is possible to easily supply, on a large scale, an alcohol concentration detecting sensor in which the electrostatic capacitance Cₛ can be increased so that the excellent result of the measurement can be obtained. Furthermore, the sensor itself is thin, very small and compact, and can be installed everywhere so that the degree of freedom of a design can be increased.

While the preferred examples of the present invention have been described above, the present invention is not restricted thereto. For example, while the description has been given to the case in which the alcohol concentration in the gasoline is detected in the examples, the present invention can also be applied to the case in which the concentration of alcohol in another liquid to be inspected is to be detected. Thus, various changes can be made without departing from the scope of the present invention.

(Advantage of the Invention)

According to the present invention, it is possible to reduce a distance between the electrodes by using the electrode wiring pattern formed on the base material resin film. As is apparent from the Equation 2, therefore, the electrostatic capacitance Cₛ can be increased so that the excellent result of the measurement can be obtained.

In addition, the alcohol concentration detecting sensor is constituted by the base material resin film, the electrode wiring pattern formed on the base material resin film, and the insulating resin covering the surface of the electrode wiring pattern. Therefore, the sensor itself is flexible, thin, very small and compact, and can be installed everywhere so that the degree of freedom of a design can be increased.

Furthermore, the surface of the electrode wiring pattern is covered with the insulating resin. Therefore, an insulation between the electrodes is excellent and is not influenced by a moisture, and shielding can be carried out in order to prevent the influence of an electromagnetic wave generated from the body of a car or the like, and furthermore, an accurate measurement for the alcohol concentration can be executed.

According to the present invention, moreover, it is possible to obtain an electrode wiring pattern having a very small distance between the electrodes, for example, within a range of approximately 5 μm to 50 μm by etching. Therefore, the electrostatic capacitance Cₛ can be increased so that the excellent result of the measurement can be obtained.

According to the present invention, furthermore, it is possible to reduce a distance between the electrodes by using the electrode wiring pattern formed on the substrate. As is apparent from the Equation 2 described above, therefore, the electrostatic capacitance Cₛ can be increased so that the excellent result of the measurement can be obtained.

In addition, the alcohol concentration detecting sensor is constituted by the substrate, the electrode wiring pattern formed on the substrate, and the insulating coat covering the surface of the electrode wiring pattern. Therefore, the sensor itself is thin, very small and compact, and can be installed everywhere so that the degree of freedom of a design can be increased.

Moreover, the surface of the electrode wiring pattern is covered with the insulating coat. Therefore, an insulation between the electrodes is excellent and is not influenced by a moisture, and shielding can be carried out in order to prevent the influence of an electromagnetic wave generated from the body of a car or the like. Furthermore, an accurate measurement for the alcohol concentration can be executed.

According to the present invention, furthermore, it is possible to obtain an electrode wiring pattern having a thickness of 0.1 to 5 μm by sputtering at a very small distance between the electrodes, for example, within a range of approximately 5 μm to 50 μm by the sputtering. Therefore, the electrostatic capacitance Cₛ can be increased so that the excellent result of the measurement can be obtained.

According to the present invention, moreover, it is possible to obtain, by chemical vapor deposition (CVD), a very minute and thin insulating coat which is not influenced by a liquid to be inspected such as a gasoline or alcohol, for example, SiO₂, Al₂O₃ or the like. Thus, the sensor itself can be thin, very small and compact.

Furthermore, the electrode does not directly come in contact with the liquid to be inspected such as a gasoline. Therefore, a defective operation can be prevented from being caused by a deterioration with the passage of time, foreign matters in the gasoline or the like. Thus, it is possible to detect the alcohol concentration accurately and rapidly.

In addition, the substrate is provided. Therefore, it is easy to assemble and attach the alcohol concentration detecting sensor into the apparatus.

According to the present invention, moreover, the positive and negative electrodes which are comb-toothed are formed to be alternately intricate. Therefore, the electrodes having a very small distance therebetween can be provided to be compact as a whole.

Accordingly, it is possible to obtain an electrode wiring pattern having a very small distance between the electrodes, for example, within a range of approximately 5 μm to 50 μm by sputtering, respectively. Therefore, the electrostatic capacitance Cₛ can be increased so that the excellent result of the measurement can be obtained.

According to the present invention, furthermore, the alcohol concentration in the gasoline can be detected accurately and rapidly. Moreover, it is possible to control a torque to be constant by excessively adding a gasoline corresponding to the amount of addition of alcohol, for example, ethanol as an antiknocking agent. Thus, the present invention can produce many remarkable and peculiar functions and advantages, which is very excellent.

1. An alcohol concentration detecting apparatus in which an alcohol concentration in the liquid to be inspected is detected by introducing a liquid to be inspected between electrodes of an alcohol concentration detecting sensor and by measuring a change in a specific inductive capacity of the liquid to be inspected between the electrodes with an oscillation frequency,
wherein the alcohol concentration detecting sensor comprises an alcohol concentration detecting sensor body including a base material resin film, an electrode wiring pattern formed on the base material resin film, and an insulating resin covering a surface of the electrode wiring pattern.

2. The alcohol concentration detecting apparatus according to claim 1, wherein the alcohol concentration detecting sensor body is stuck onto a substrate.

3. The alcohol concentration detecting apparatus according to claim 1, wherein the electrode wiring pattern is obtained by selectively etching a conductive metallic foil laminated on one of surfaces of the base material resin film, thereby forming a wiring pattern taking a predetermined shape.

4. The alcohol concentration detecting apparatus according to claim 1, wherein the electrode wiring pattern has such a shape that positive and negative electrodes which are comb-toothed are alternately intricate.

5. An alcohol concentration detecting apparatus in which an alcohol concentration in the liquid to be inspected is detected by introducing a liquid to be inspected between electrodes of an alcohol concentration detecting sensor and by measuring a change in a specific inductive capacity of the liquid to be inspected between the electrodes with an oscillation frequency,
wherein the alcohol concentration detecting sensor comprises a substrate, an electrode wiring pattern formed on the substrate, and an insulating coat covering a surface of the electrode wiring pattern.

6. The alcohol concentration detecting apparatus according to claim 5, wherein the electrode wiring pattern is obtained by selectively etching a conductive metallic thin film formed on one of surfaces of the substrate by sputtering, thereby forming a wiring pattern taking a predetermined shape.

7. The alcohol concentration detecting apparatus according to claim 5, wherein the insulating coat is formed by chemical vapor deposition (CVD).

8. The alcohol concentration detecting apparatus according to claim 5, wherein the electrode wiring pattern has such a shape that positive and negative electrodes which are comb-toothed are alternately intricate.

9. An alcohol concentration detecting method of detecting an alcohol concentration in a liquid to be inspected by using the alcohol concentration detecting apparatus according to claim 1,
wherein an alcohol concentration in the liquid to be inspected is detected by introducing a liquid to be inspected between electrodes of an alcohol concentration detecting sensor and by measuring a change in a specific inductive capacity of the liquid to be inspected between the electrodes with an oscillation frequency.

10. An alcohol concentration detecting method according to claim 9, wherein the liquid to be inspected is a gasoline containing alcohol.

11. A method of manufacturing an alcohol concentration detecting sensor, comprising:
a conductive metallic foil sticking step of sticking a conductive metallic foil onto one of surfaces of a base material resin film;
a photoresist applying step of applying a photoresist onto a whole upper surface of the conductive metallic foil;
a photoresist exposing step of exposing the photoresist to take a desirable electrode wiring pattern shape by using a photoresist mask;
a photoresist dissolving and removing step of dissolving and removing the exposed photoresist portion with a developing solution;
an etching step of etching and removing a conductive metallic foil portion which is not covered with the photoresist, with an etchant;
a photoresist dissolving and removing step of dissolving and removing the photoresist; and
an insulating resin applying step of applying an insulating resin onto the surface from which the photoresist is removed, thereby obtaining an alcohol concentration detecting sensor body.

12. The method of manufacturing an alcohol concentration detecting sensor according to claim 11, further comprising a substrate sticking step of sticking the alcohol concentration detecting sensor body obtained at the insulating resin applying step, onto a substrate.

13. The method of manufacturing an alcohol concentration detecting sensor according to claim 11, wherein the base material resin film is a polyimide resin film.

14. The method of manufacturing an alcohol concentration detecting sensor according to claim 11, wherein the conductive metallic foil is a copper foil.

15. The method of manufacturing an alcohol concentration detecting sensor according claim 11, wherein the insulating resin is constituted by at least one selected from a urethane resin, a polyimide resin and n epoxy type resin.

16. The method of manufacturing an alcohol concentration detecting sensor according to claim 11, wherein the electrode wiring pattern has such a shape that positive and negative electrodes which are comb-toothed are alternately intricate.

17. A method of manufacturing an alcohol concentration detecting sensor, comprising:
a conductive metallic thin film forming step of forming a conductive metallic thin film on one of surfaces of a substrate by sputtering;
a photoresist applying step of applying a photoresist onto a whole upper surface of the conductive metallic thin film;
a photoresist exposing step of exposing the photoresist to take a desirable electrode wiring pattern shape by using a photoresist mask;
a photoresist dissolving and removing step of dissolving and removing the exposed photoresist portion with a developing solution;
an etching step of dry etching and removing a conductive metallic thin film portion which is not covered with the photoresist;
a photoresist dissolving and removing step of dissolving and removing the photoresist; and
an insulating coat forming step of forming an insulating coat on a surface of the electrode wiring pattern from which the photoresist is removed, by chemical vapor deposition (CVD),

18. The method of manufacturing an alcohol concentration detecting sensor according to claim 17, wherein the substrate is constituted by at least one selected from ceramics, glass and a resin substrate.

19. The method of manufacturing an alcohol concentration detecting sensor according to claim 17, wherein the conductive metallic thin film is constituted by at least one selected from platinum, nickel, copper and titanium.

20. The method of manufacturing an alcohol concentration detecting sensor according to claim 17, wherein the insulating coat is constituted by at least one minute insulating coat selected from SiO₂, Al₂O₃ and the like.

21. The method of manufacturing an alcohol concentration detecting sensor according to claim 17, wherein the electrode wiring pattern has such a shape that positive and negative electrodes which are comb-toothed are alternately intricate.
